(19) United States
(12) Patent Application Publication
(10) Pub. No.: US 2009/0312666 A1
(43) Pub. Date: Dec. 17, 2009

(54) SKIN CONDUCTION MEASURING APPARATUS

(76) Inventors: Takenori Fukumoto, Kanagawa (JP); Hisashi Akiyama, Kanagawa (JP)

Correspondence Address: WENDEROTH, LIND & PONACK L.L.P., 1030 15th Street, N.W., Suite 400 East, Washington, DC, 20005-1503, US

(21) Appl. No.: 12/307,807

(22) PCT Filed: Jul. 9, 2007
(86) PCT No.: PCT/JP2007/063664

§ 371 Date: Jan. 7, 2009

(30) Foreign Application Priority Data
Jul. 10, 2006 (JP) 2006-189203

(51) Int. Cl. A61B 5/053

(52) U.S. Cl. 600/547

(57) ABSTRACT

In this skin conduction measuring apparatus, bipolar pulse currents generated by current generator sections 11a to 11i are applied to plural measurement points of a skin 30 of a subject through nonpolarizable electrodes 3a to 3i. The conducted currents and voltages generated by the conduction are measured by a measuring section 6. A feature quantity that characterizes current conductivity at each of the measurement points is extracted by a feature quantity extracting section 7 and then the result is displayed by a display section 8. An index extracted in the feature quantity extracting section 7 is calculated based on electrical equivalent circuit parameters Rp, Cp, and Rs of the skin 30. Quantitative measurement results with sufficient reliability and reproducibility can be obtained.

### TECHNICAL FIELD

The present invention relates to a skin conduction measuring apparatus to be used for Ryodoraku medicine in which an electric current conductivity of the human body is measured for searching positions of acupuncture points and for evaluating a health level.

### BACKGROUND ART

There have conventionally been proposed techniques related to skin conduction measuring apparatuses to be used for Ryodoraku medicine, in which electrical conductivities of specific points on a living body are measured for searching positions of acupuncture points or for evaluating the health level or the like based the measurement results (e.g., JP 2003-61926 A and JP H9-75419 A). In these prior arts, with a DC voltage applied between two metal electrodes placed on a skin surface of a subject at specific sites, a DC current flowing through between the two electrodes is measured to thereby measure electrical conductivities in direct current at the specific sites. The “acupuncture point” exists as therapeutic point in oriental traditional medicine. By applying a physical stimulation (e.g., mechanical, thermal, or electrical stimulation) to the acupuncture points, elimination of pains or control of an autonomic nervous system can be achieved. Most of the acupuncture points are in many cases observed as sites of lower electrical skin resistance as compared with peripheral sites, and those low-resistance sites of the skin are known to be distributed along a “meridian (in brief, a line interconnecting acupuncture points).” That is, while the low-resistance sites of the skin and the acupuncture points are regarded as equivalent to each other, it is practiced to search such sites by the skin conduction measuring apparatus and stimulate them for curing. These actions are called as “ryodoraku autonomic nerve system therapy.” The description herein is also based on the assumption where the low-resistance sites of the skin and the acupuncture points are equivalent.

FIG. 6 outlines a measuring apparatus which embodies the invention disclosed in JP 2003-61926 A. An electrode of a hand-grip probe 201 is a bar-like member made of metal and a user performs measurement while gripping the hand-grip probe 201 by one hand and gripping a measurement probe 203 by the other hand. Provided at an end of the measurement probe 203 is a cone-shaped cap 207 with a metallic electrode member (not shown) placed inside. For measurement, wetted cotton is filled in the cap 207 so as to be in contact with the electrode member of the measurement probe 203, and the cotton is applied to a measurement site. Thereafter, a DC current that has flowed through a range of the living body between the probes 201 and 203 gripped by both hands due to a DC voltage Ec applied from a variable DC voltage source 202 is converted into a voltage value by a detection resistor 206. In FIG. 6, a reference numeral 204 denotes a variable resistor for current adjustment and a reference 208 denotes a capacitor for balancing.

### DISCLOSURE OF THE INVENTION

### Problem to be Solved by the Invention

Described below is a detailed investigation on the prior art skin conduction measuring apparatus made by the inventors of the present invention.

In the prior art of FIG. 6, an electrical equivalent circuit of electrodes of the hand-grip probe 201 and the measuring director 203, and an electrical equivalent circuit of the skin, even simply considered as it is, result in a circuit in which a parallel-connected circuit including a resistor 801 of a resistance value Rp and a capacitor 802 of a capacitance Cp is connected in series with a resistor 803 of a resistance value Rs as shown in FIG. 7. Also, it is known that an electrical equivalent circuit of deep tissues of the living body can be represented in a form that resistors are connected in series with one another. Therefore, an equivalent circuit for measurement by the measuring apparatus of FIG. 6 can be represented by such a circuit as shown in FIG. 8.

In FIG. 8, for simplicity, an electrode 201a of the hand-grip probe 201 and an electrode 203a of the measurement probe 203 are respectively placed at points A and B on the skin. An electrical equivalent circuit of the electrode 203a of the measurement probe 203 is so formed that a parallel-connected circuit of a resistor 301 (resistance Re1) and a capacitor 302 (capacitance Ce1) is connected in series to a resistor 303 (resistance Res1). Similarly, the electrical equivalent circuit of the electrode 201a of the hand-grip probe 201 is so formed that a parallel-connected circuit of a resistor 401 (resistance Re2) and a capacitor 402 (capacitance Ce2) is connected in series to a resistor 403 (resistance Res2). An electrical equivalent circuit of the skin to be in contact with the electrodes 203a and 201b, respectively, of the measurement probe 203 and hand-grip probe 201 is so formed that a parallel-connected circuit of resistors 501, 601 (resistances Rs1, Rs2) and capacitors 502, 602 (capacitances Cs1, Cs2) is connected in series to resistors 503, 603 (resistances R1, R2). A plurality of series-connected resistors 703 (the resistors 503, 603 are also connected in series with these resistors 703) constituting an equivalent circuit of the deep tissue have resistance values Ri (i=1 to N). Impedances of the equivalent circuits of the electrodes 201a, 203a and skins to be in contact with these electrodes 201a, 203a are respectively assumed as Ze1, Ze2, Zs1 and Zs2.

In the prior art of FIG. 6, a DC current Ic during application of the DC voltage Ec is measured, which means that only a DC resistance of the equivalent circuits of FIG. 8 is considered. That is, the DC current Ic expressed by the following Equation (1) is measured.

$${I}_{C} = \frac{{E}_{c}}{\begin{matrix}{\sum }_{i = 1}^{N} {R}_{i} + {R}_{e 1} + {R}_{es 1} + {R}_{s 1} + \\ {R}_{e 2} + {R}_{es 2} + {R}_{s 2} + {R}_{c} + {R}_{va}\end{matrix}} \qquad ( 1 )$$

In Equation (1), the resistance Rc of the detection resistor 206 and the resistance Rva of the adjusting resistor 204 are known values. Accordingly, measuring the current Ic of Equation (1) is equivalent to detecting variation in the resistance values of the resistors other than the resistors 206 and 204 depending on the measurement sites or measurement time. This conventional measurement method can not sufficiently ensure reliability and reproducibility of measurement results. This is mainly because of the following four reasons:
(1) A two-electrode method are applied for measurement;
(2) The DC resistance in the electrical equivalent circuit of the skin is only considered;
(3) Polarizable electrodes are used; and
(4) A Voltage or current dependency of the skin resistance is not taken into consideration.

These reasons (1) to (4) are described in detail below.

First, as to the reason (1), the current measured as described above is expressed by Equation (1). The resistances Rc and Rva are known values that can be externally controlled. Differences of the measured current are due to differences of electrical property expressed by the following Equation (2) between the electrode 201a (point A) and the electrode 203a (point B) and therefore the measured current is not the current value due to the skin resistance between the two electrodes 201a and 203a in a precise sense.

$${\sum }_{i = 1}^{N} {R}_{i} + {R}_{e 1} + {R}_{es 1} + {R}_{s 1} + {R}_{e 2} + {R}_{es 2} + {R}_{s 2} \qquad ( 2 )$$

If the impedances of the two electrodes 201a and 203a are sufficiently smaller than that of the living body, that is, if Ze1<<Zs1 and Ze1<<Zs1 in FIG. 8, i.e., if it is satisfied that (Re1+Res1)<<(Rs1+R1) and (Re2+Res2)<<(Rs2+R2), the skin resistance can be properly evaluated. However, it is generally known that an electrode impedance of a metal electrode increases with decreasing frequency and that polarizable electrodes as will be described later involve extremely large values of DC resistances. As a result, the two-electrode method is incapable of properly evaluating the skin resistance. Further, even if it is supposed that electrode impedances are small, it is impossible to discriminate between a difference due to the DC resistance of skin immediately under the electrode 201a (point A) and a difference due to the DC resistance of skin immediately under the electrode 203a (point B). In spite of that it is essentially intended to measure the differences of the current values due to the skin resistance at the point B immediately under the electrode 203a of the measuring director 203, yet it is impossible to clearly discriminate which of the two electrodes 201a and 203a is the one associated with the current value difference due to a DC resistance of skin immediately under the electrode.

As to the reason (2), if the electrical equivalent circuit of the skin can be represented only by the pure DC resistors, then a current waveform obtained from measurement by the prior art measuring apparatus is as shown by one-dot chain line in FIG. 9B. The one-dot chain line shows that a steady state is reached immediately after contact of the electrode with the skin (FIG. 9A shows a waveform of the applied DC voltage Ec). However, because the electrical equivalent circuit of the skin generally comprises the parallel connection of the resistors 501, 601 and the capacitors 502, 602 as described before, the measured current involves a transient response as shown by solid line in FIG. 9B. For dissipating the transient response and achieving the steady state, a time duration of four times (4τ) the time constant τ (=Rp·Cp) is required even in the case where the electrical equivalent circuit of the skin is represented by such a simplest circuit as shown in FIG. 7. This means that even if measurement objects have same characteristic, measurement results differ depending on the time at which the measured current value is read until elapsed time becomes 4τ or more. Further, in case of the skin of the living body, it is predicted that the value of time constant τ differs to a large extent depending on the measurement site. Therefore, even if differences in current value among a plurality of sites are measured for a constant measuring time duration, it cannot be ensured that the measured current have already reached the steady state at every measurement point. However, measuring the current values after a long-time elapse causes disadvantageous that the measurement requires long time and irreversible changes in characteristics of the skin and electrode due to that unidirectional voltage or current is applied for a long time, such irreversible changes including an electrical damage to the skin and start of electrolysis of the electrodes. Meanwhile, even with a very small time constant τ and with a steady state provided immediately after the start of measurement, there are some cases where the actually measured current waveforms vary. The reason for this is that the ion concentration differences at interfaces between respective two electrodes and the skin are inconstant. The reason is common for that when electrodes are placed at two points on the skin, measuring the voltage between those points involves spontaneous irregular fluctuations of voltage from several millivolts to several hundreds of millivolts. In particular, the human palm involves large fluctuation in the ion concentration at the interface between the skin and electrodes due to mental sweating or the like, so that such the conventional measurement method as described above would result in unstable measurement results, which would lead to dependency of a measurement result on the selection of a time point.

As to the reason (3), inactive polarizable electrodes such as platinum electrodes have a noticeable nonlinearity of voltage versus current characteristics due to limited mobility of electric charges on a surface of such electrodes. Further, because of large electrode resistances corresponding to the resistance values Rp (in FIG. 8, resistances Re1, Re2 of resistors 301, 401) of the resistors connected in parallel with the capacitor in the equivalent circuit of the polarizable electrode, the material of electrodes used or the values of voltage or current to be applied can cause magnitude relationships between the impedances Ze1, Zs1 and Ze2, Zs2 in FIG. 8 to be that Ze1>>Zs1 or Ze2>>Zs2. This means that it cannot be discriminated whether, with use of polarizable electrodes, the measurement is for measurement of the characteristics of the skin or for measurement of differences due to electrode characteristics.

As to the reason (4), an electrical characteristic of a biogenic tissue such as the skin has current or voltage dependency for similar reason as the current or voltage dependency of the electrode described above regarding the reason (3). Generally, with a small value of current or voltage to be applied and with a high frequency, the dependency does not matter and the electrical characteristic of the skin can be regarded as linear. However, the lower the frequency is, or the larger the current value or voltage value is, the more noticeable the nonlinearity becomes. The prior art described above gives no consideration to this nonlinearity. Further, in terms of the degree of this nonlinearity, it is known that conditions under which the nonlinearity occurs vary among individual measurement objects and measurement sites. Therefore, even with a constant value of applied voltage or current used for the measurement, a noticeable nonlinearity may be involved depending on the measurement site, making it difficult to ensure the reliability of measurement results.

As described above, the present inventors have found out anew that the above prior art measurement method has such many measurement problems as to be incapable of sufficiently ensuring the reliability and reproducibility of measurement results.

The present invention is intended to avoid as much as possible such problems of the prior art as described above. Thus, an object of the invention is to provide a skin conduction measuring apparatus having reliability and reproducibility by virtue of its measuring technique in which enough considerations are given to electrical characteristics of the skin.

### Means for Solving the Problem

In order to solve the above problems of the prior art, the present invention provides a skin conduction measuring apparatus comprising: a current generator section capable of generating pulsed electric currents; an electrode system having a plurality of nonpolarizable electrodes to be placed on a plurality of different measurement points on a skin and functioning for conducting the currents output from the current generator section to the plurality of measurement points substantially simultaneously (or without delay); a plurality of current detectors for respectively detecting the currents conducted to the plurality of measurement points; a measuring section for measuring the currents detected by the current detectors and for measuring voltages in the skin at the plurality of measurement points generated by the conduction of the electrode system; a feature quantity extracting section for extracting a feature quantity that characterizes an electric current conductivity at each of the measurement points from a relationship between the current and the voltage measured by the measuring section; a display section for displaying the feature quantity at each of the measurement points extracted by the feature quantity extracting section; and a control section for generating control signals for the current generator section, the measuring section, and the feature quantity extracting section.

This arrangement ensures measurement results with enough reliability and reproducibility as compared with the prior art.

Further, the skin conduction measuring apparatus according to the present invention is characterised in that the nonpolarizable electrodes are silver-silver chloride electrodes.

This arrangement minimizes effects of electrode impedances on the measurement results. Further, the nonpolarizable electrodes may have a solid gel or paste containing an electrolyte.

Further, the skin conduction measuring apparatus according to the present invention is characterised in that the pulsed electric currents generated by the current generator section are bipolar pulse currents.

This arrangement can makes a net charge to the living body during the measurement zero, thereby avoiding irreversible changes in characteristics of the electrodes and a living body.

In the skin conduction measuring apparatus according to the present invention it is preferable that the control section sets current values of the currents output from the current generator section to different values for the plurality of measurement points.

This arrangement achieves proper adjustment of stimulation quantities, resulting in that effective stimulation can be given to the living body with less quantities of stimulation.

Especially, it is preferable that the control section sets the currents output from the current generator section to such values that current dependency of the skin at the measurement points is not observed

This arrangement avoids irreversible changes in characteristics of the electrodes and the living body

Further, the skin conduction measuring apparatus according to the present invention is characterised in that the feature quantity extracted by the feature quantity extracting section is associated with at least two of a resistance value Rp of a first resistor, a capacitance Cp of a capacitor, and a resistance value Rs of a second resistor under an assumption where an electrical equivalent circuit of the skin consists of the first resistor and the capacitor parallel-connected to each other and the second resistor series-connected to the parallel-connected first resistor and the capacitor.

This arrangement can provides quantitative measurement results more reliable than those obtained by the prior art.

Furthermore, the skin conduction measuring apparatus according to the present invention is characterised in that the feature quantity extracted by the feature quantity extracting section is an electrical conductivity G having a relation with the resistance value Rp and the resistance value Rs defined by the following Equation (3).

G=1/(Rp+Rs) (3)

This arrangement can provides quantitative measurement results more reliable than those obtained by the prior art.

Further, the skin conduction measuring apparatus according to the present invention is characterised in that the feature quantity extracted by the feature quantity extracting section is a time constant τ having a relation with the resistance value Rp and the capacitance Cp defined by the following Equation (4).

r=1/(Rp·Cp) (4)

This arrangement can provides quantitative measurement results more detail and reliable than those obtained by the prior art.

Preferably, the control section sets the currents values of the currents output from the current generator section respectively for the plurality of measurement points based on the feature quantities respectively extracted by the feature quantity extracting section.

This arrangement achieves proper adjustment of stimulation quantities, resulting in that effective stimulation can be given to the living body with less quantities of stimulation.

### EFFECT OF THE INVENTION

According to the present invention, the above-described characteristics achieves more proper evaluation of a skin conduction and thus the skin conduction measuring apparatus can obtain more detail, quantitative, reliable, and reproducible measurement results.

### BRIEF DESCRIPTION OF THE DRAWINGS

FIG. 1 is a block diagram showing an outlined construction of a first embodiment of the present invention;

FIG. 2A is a block diagram showing details of part of the skin conduction measuring apparatus of the first embodiment of the present invention;

FIG. 2B is a block diagram showing details of part of the skin conduction measuring apparatus in the first embodiment of the present invention;

FIG. 3 is a schematic chart of a conduction current waveform and a voltage waveform in the first embodiment of the present invention;

FIG. 4 is a schematic chart in which the voltage waveform is partly enlarged;

FIG. 5 is a schematic chart for explaining operation of extracting feature quantities in a second embodiment of the present invention;

FIG. 6 is a schematic diagram showing a skin conduction measuring apparatus according to a prior art;

FIG. 7 is a schematic diagram of an electrical equivalent circuit of skin;

FIG. 8 is a schematic diagram for explaining problems of the prior art;

FIG. 9A is a schematic chart showing a voltage waveform for explaining a problem of the prior art; and

FIG. 9B is a schematic chart showing a current waveform for explaining a problem of the prior art.

### DESCRIPTION OF REFERENCE SIGNS

### Best Mode for Carrying Out the Invention

Hereinbelow, embodiments of the present invention will be described with reference to the accompanying drawings.

### First Embodiment

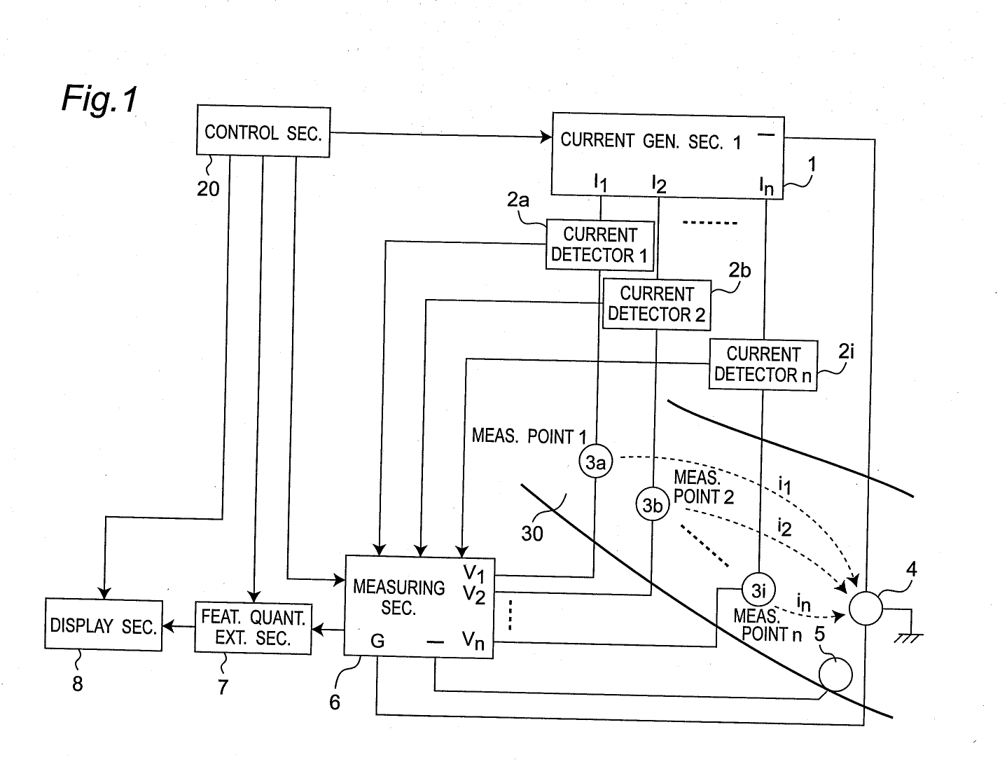

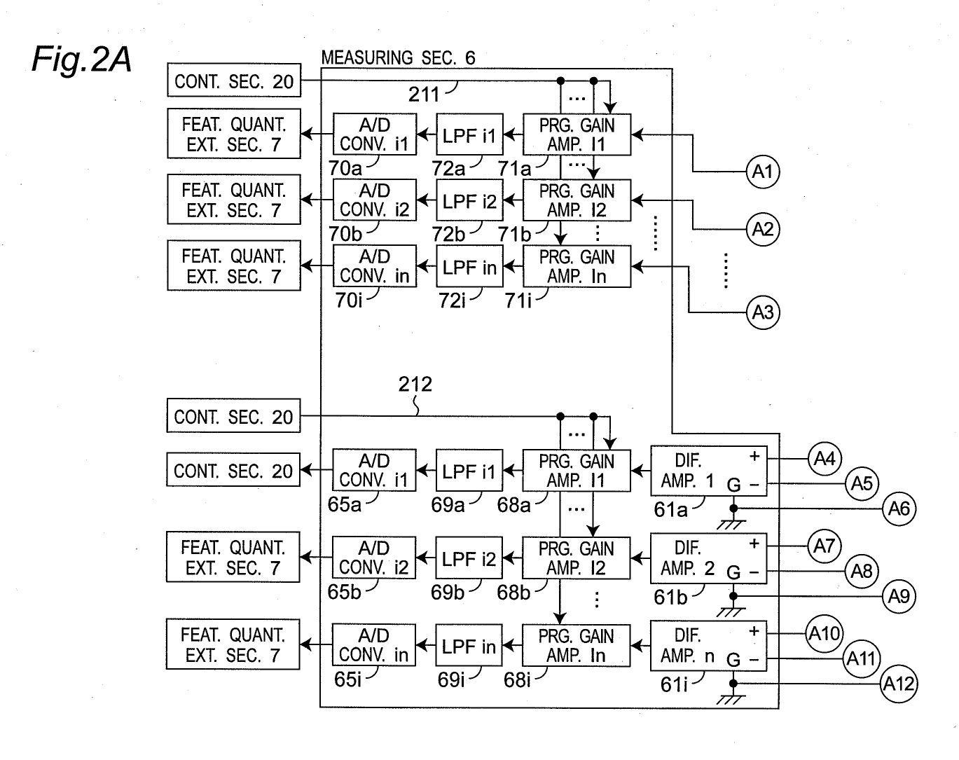

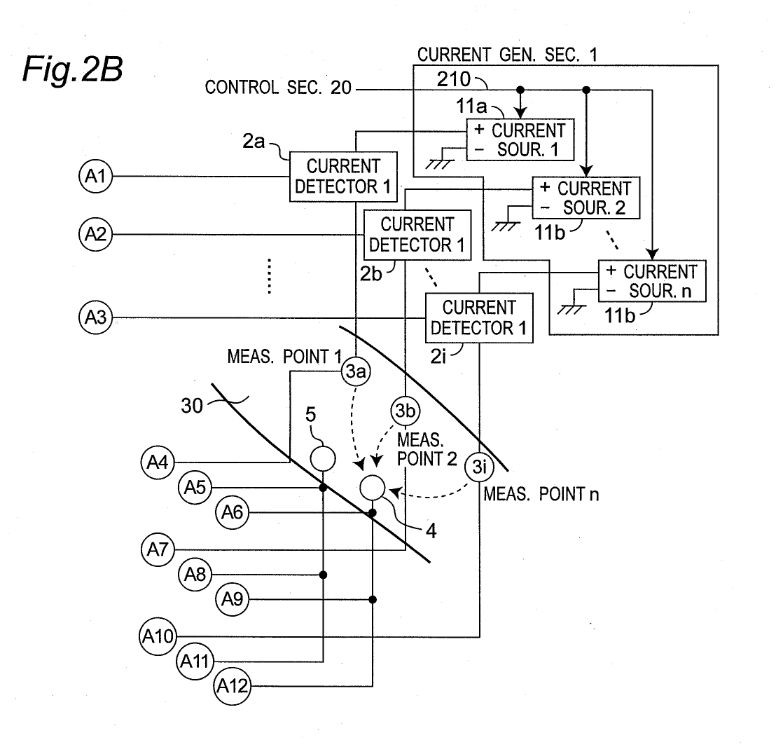

FIG. 1 is a block diagram showing an outlined construction of a skin conduction measuring apparatus according to a first embodiment of the present invention. FIGS. 2A and 2B are block diagrams showing details of examples of a current generator section 1 and measuring section 6. This skin conduction measuring apparatus comprises a current generator section 1, an electrode system including a plurality of electrodes 3a to 3i, 4, and 5, current detectors 2a to 2i, a measuring section 6, a feature quantity extracting section 7, a display section 8, and a control section 20. The current generator section 1 has at least one or more current sources 1 to “n”. The current system includes at least one or more current-applying electrodes 3a to 3i, a ground electrode 4, and a indifferent electrode 5. The measuring section 6, for measurement of voltages and processing of voltage measured values, includes at least one or more differential amplifiers 61a to 61i, programmable gain amplifiers 68a to 68i, low-pass filters 69a to 69i, and at least one or more A/D converters 65a to 65i. Further, the measuring section 6, for measurement of currents and processing of current measured values, includes programmable gain amplifiers 71a to 71i, low-pass filters 72a to 72i, and A/D converters 70a to 70i. The control section 20 generates control signals for the current generator section 1, the measuring section 6, the feature quantity extracting section 7, and the display section 8.

The current-applying electrodes 3a to 3i are respectively connected to their corresponding current sources 1 to “n” of the current generator section 1. The current detectors 2a to 2i are respectively interposed between the current-applying electrodes 3a to 3i and the current sources 1 to “n”. Further, the current-applying electrodes 3a to 3i are respectively connected to their corresponding differential amplifiers 61a to 61i of the measuring section 6. The indifferent electrode 5 of the electrode system is connected to the differential amplifiers 61a to 61i of the current generator section 1.

Currents generated by the current generator section 1 are applied to measurement points (measurement point 1 to measurement point “n”) of a skin 30 of a subject through the current-applying electrodes 3a to 3i and then flows to the ground electrode 4. Voltage drops caused by this electric conduction in the skin between the individual current-applying electrodes 3a to 3i and the indifferent electrode 5 are measured by the differential amplifiers 61a to 61i of the measuring section 6 by referencing a potential of the ground electrode 4. A technique of performing measurement by such an electrode system is called three-electrode method, by which skin impedances immediately under the current-applying electrodes 3a to 3i, i.e. immediately under the measurement points 1 to “n”, are measured.

In this embodiment, all of the electrodes 3a to 3i, 4, and 5 shown in FIG. 1 are nonpolarizable electrodes. For example, Ag—AgCl (silver-silver chloride) electrodes can be used as the electrodes 3a to 3i, 4, and 5. By adopting the nonpolairzable electrodes, an electrode impedance Ze and a skin impedance Zs normally satisfy a relationship that Zs>>Ze so that measured currents constantly results from differences or variations of the skin impedances Zs. This makes it possible to evaluate skin resistances more properly as compared with the prior art adopting polarizable electrodes. Although this embodiment is described based on the use of nonpolarizable electrodes because usage of the nonpolarizable electrodes can easily satisfy the Zs>>Ze, polarizable electrodes of relatively low polarization resistance such as those of Ag (silver) can be used on the condition that Zs>>Ze is satisfied. Further, in order to maintain an electrically-good contact state with the skin 30, an electrolyte-containing solid gel or paste processed so as to have an area similar to the electrode area is placed between the electrodes 3a to 3i, 4, 5 and the skin 30. It should be noted that use of the solid gel is more preferable than the paste because using the paste might cause drastic changes in electrical characteristics of the skin with time due to moisture contained in the paste.

The current sources 11a to 11i of the current generator section 1 generate currents conducted to the respective measurement points. In this embodiment, amplitude, cycle period and number of cycles of bipolar pulse currents generated by the current sources 11a to 11i can be set by a control signal 210 from the control section 20. The current values of the currents conducted from the current sources 11a to 11i to the individual measurement points are set so that current dependency is not observed in the skin 30 at each of the measurement points. Whereas various techniques are available for conduction of current values showing no current dependency, one example of simple techniques for this purpose is as follows. While pulse currents conducted from the individual current sources 11a to 11i of FIG. 2A are gradually increased in value from zero, voltage waveforms resulting from the conduction are measured by the measuring section 6. Then, results of dividing the measured voltage waveforms by conducted pulse current values are superimposed. If the current dependency does not exist, the divided voltage waveforms for different pulse current values have the same waveform profile. Thus, the smallest current value that causes different waveform profiles among the divided voltage waveforms is detected. A current value half the detected smallest current value is used for the measurement. These sequence is applied to the individual measurement points.

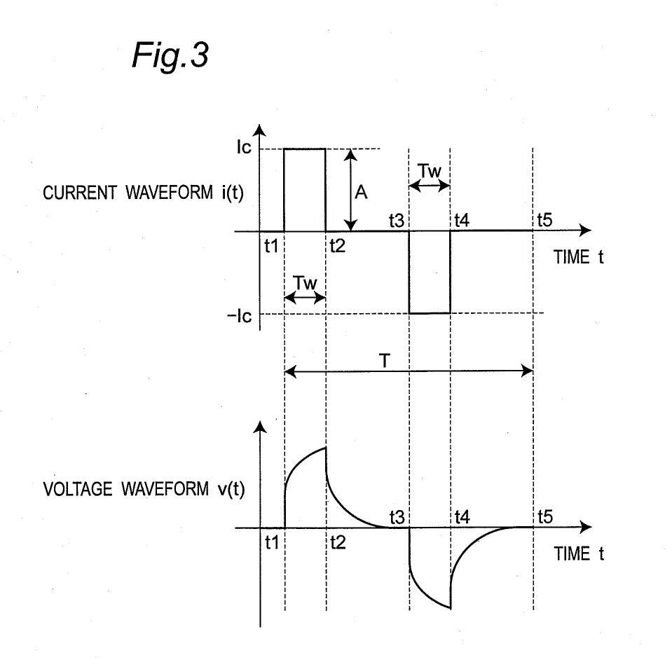

FIG. 3 shows a schematic chart of the bipolar pulse current waveform i(t) and a voltage waveform v(t) generated on the skin by the conduction of the bipolar pulse current waveform i(t). FIG. 3 shows a case where the skin 30 is represented by the equivalent circuit shown in FIG. 7 described before. In FIG. 3 reference character “t1” denotes conduction start time, i.e. positive rising-edge time, “t2” denotes falling-edge time from the positive to zero, “t3” denotes negative falling-edge time, “t4” denotes rising-edge time from the negative to zero, “t5” denotes conduction end time, “A” denotes pulse amplitude, “Tw” denotes pulse width, and “T” denotes pulse period. The schematic chart of FIG. 3 shows a case where one bipolar pulse current having the period “T” is conducted. However, the present invention is not limited to such case and a plurality of pulses each of which is as shown in FIG. 3 may be conducted.

The Voltages generated at the skin 30 of the measurement points 1 to “n” by electric conduction are respectively measured by the differential amplifiers 61a to 61i. The measured voltages of the measurement points 1 to “n” are respectively amplified, as required, by the programmable gain amplifiers 68a to 68i and then subject to elimination of unnecessary high-frequency components by the low-pass filters 69a to 69i. Further, the currents conducted to the skin at the individual measurement points are respectively measured by the current detectors 2a to 2i. In view of simplification of processing by the feature quantity extracting section 7 described later, it is preferable that the same signal processing as that performed for the voltages at the individual measurement points is performed for the currents conducted to the individual measurement points. Therefore, as same as for differential amplifiers 61a to 61i, the programmable gain amplifiers 71a to 71i and the low-pass filters 72a to 72i are respectively provided for the individual current detectors 2a to 2i. Amplification factors of the programmable gain amplifiers 71a to 71i and 68a to 68i are set controllable by control signals 211 and 212 output from the control section 20.

The present invention is not limitative in terms of the sequence or means of signal processing to be performed on the measured currents and voltages. As far as desired feature quantities can be precisely obtained by the feature quantity extracting section 7, the sequence and means of signal processing are not particularly limited.

The bipolar pulse current waveforms i(t) applied to the individual measurement points and the voltage waveforms v(t) at the individual measurement points are respectively converted into digital signals by the A/D converters 65a to 65i and 70a to 70i and then fed to the feature quantity extracting section 7.

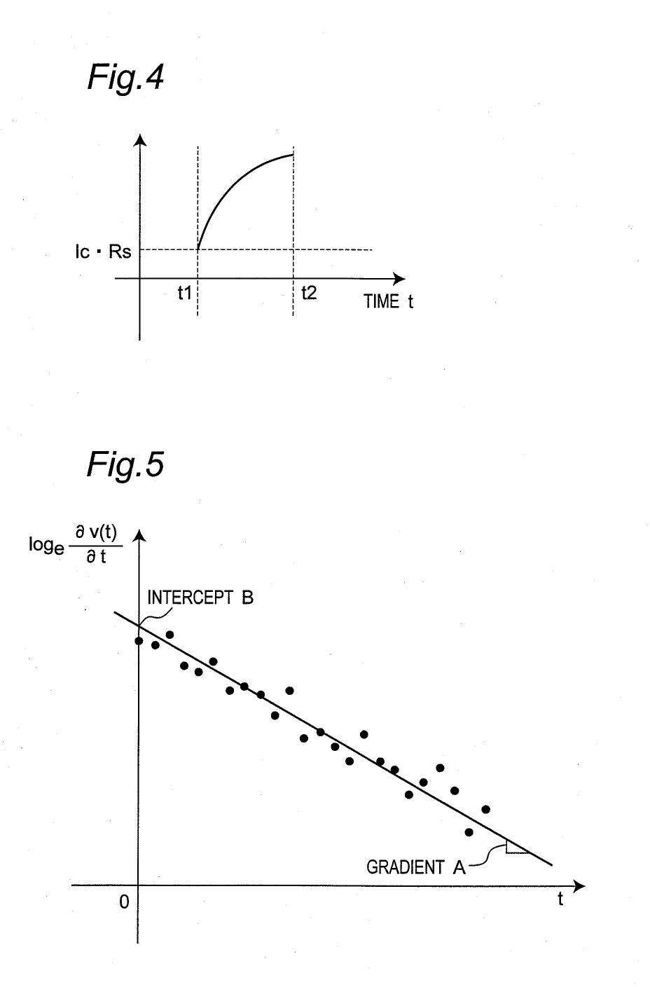

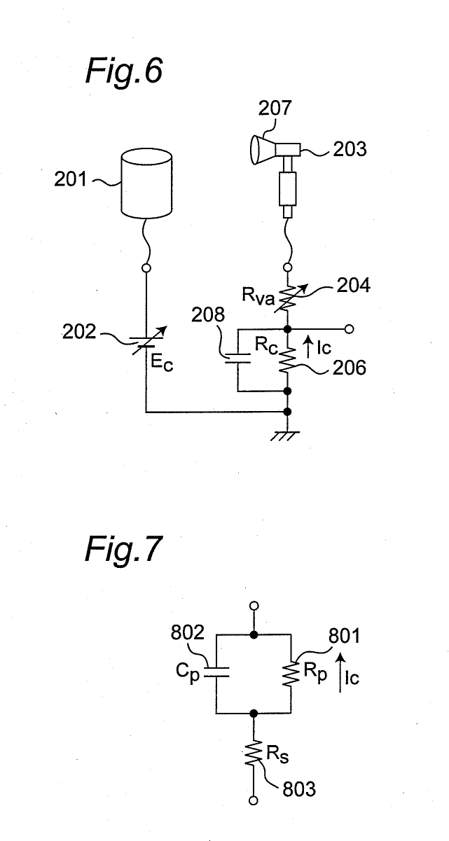

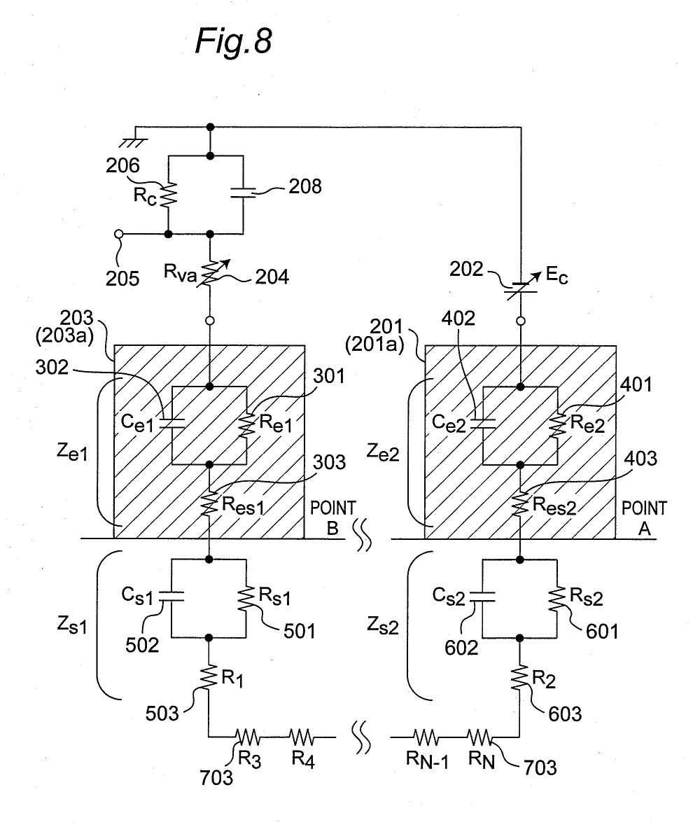

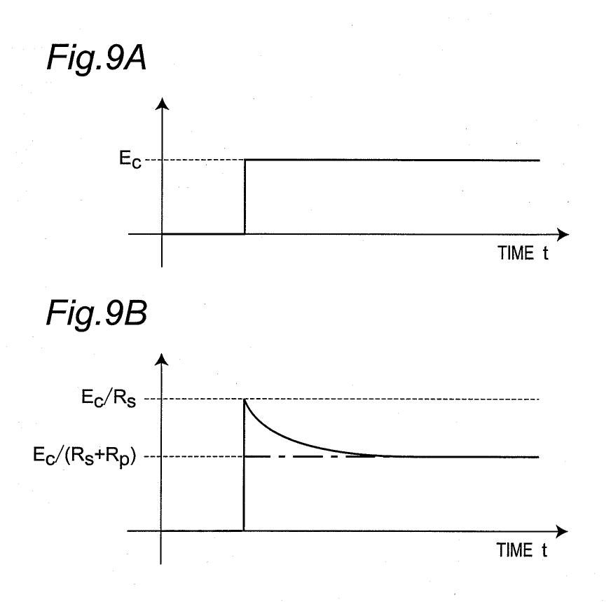

The feature quantity extracting section 7 estimates the resistance values Rp, Rs and capacitance Cp, which are parameters of the electrical equivalent circuit of the skin, from the pulse current waveforms i(t) conducted to the skin at the individual measurement points and the voltage waveforms v(t) of the skin at the individual measurement points. The estimation is based on the assumption that the electrical equivalent circuit of the skin is a simple primary system (the circuit in which the parallel-connected circuit including the resistor 801 of the resistance value Rp and the capacitor 802 of the capacitance Cp is connected in series with the resistor 803 of the resistance value Rs as shown in FIG. 7). FIG. 4 shows part (from time t1 to t2) of the voltage waveform of FIG. 3 as it is enlarged. Under the assumption that the electrical equivalent circuit of the skin 30 is represented as shown in FIG. 7, an ideal measured voltage waveform Vt(t) is expressed by the following Equation (5) in which a reference sign “Ic” denotes an amplitude of the pulse current.

$${v}_{t} ( t ) = {I}_{c} \{ {R}_{s} + {R}_{p} ( 1 - {e}^{- \frac{t}{{R}_{p} {C}_{p}}} ) \} \qquad ( 5$$

The resistance value Rs in the above equation can be calculated based on that the voltage value at t=0 can ideally be represented as Vt(0)=Ic·Rs. However, in view of that the resistance value Rs is generally smaller than the resistance value Rp and that the settling time of amplifiers has a finite value, it is difficult to precisely measure v(0). Accordingly, it is impractical to use v(0) for precise estimation of the resistance value Rs. For this reason, in this embodiment, values of v(t) measured during a time duration from t=0 to t=t1 are approximated to Vt(t) of Equation (5) by using a nonlinear least squares method such as Levenberg-Marquardt algorithm, thereby estimating the values of Rs, Rp, and Cp. Further, for calculation of the electrical conductivity G which is an index used in the prior art described before, it is enough to consider only resistance components out of the equivalent circuit of FIG. 7. Therefore the feature quantity extracting section 7 calculates the electrical conductivity G from the equation that G=1/(Rp+Rs).

Although the time duration used for the estimation is set as one from t=t1 to t=t2 in the above description, the present invention is not limited to this. For example, any time range, such as from t=t3 to t=t4, may be used for the estimation as far as that the time duration allows the values of the parameters Rs, Rp, and Cp of the equivalent circuit to be precisely estimated.

The parameter values Rs, Rp, and Cp of the equivalent circuit estimated by the feature quantity extracting section 7 as described above and the feature quantity G are fed to the display section 8 so as to be displayed as required by a monitor or other display means.

### Second Embodiment

The block diagram showing an outlined construction of a second embodiment of the invention is the same as FIG. 1 and thus same elements as those of the first embodiment are designated by same reference numerals with omitting their descriptions. This embodiment differs from the first embodiment in that the time constant τ of the equivalent circuit is extracted as the feature quantity by the feature quantity extracting section 7. Operations and other elements are omitted in description. Specific contents of the feature quantity extracting method in this embodiment will be described herebelow.

In the first embodiment, under the assumption that the electrical equivalent circuit of the skin is the simple primary system, all of the three parameters Rs, Rp, and Cp are estimated using that the response waveform Vt(t) of the equivalent circuit is ideally represented by Equation (5). The feature quantity extracting section 7 may use the results of the estimations to calculate the time constant τ as the feature quantity from the relationship that τ=1/(Rp·Cp) and then outputs the calculation result to the display section 8. However, in this embodiment, a time differential waveform of vt(t) listed below is considered.

$$\frac{\partial {v}_{t} ( t )}{\partial t}$$

This time differential coefficient is expressed from Equation (5) as shown in the following Equation (6):

$$\frac{\partial {v}_{t} ( t )}{\partial t} = \frac{{I}_{c}}{{C}_{p}} {e}^{- \frac{t}{{R}_{p} {C}_{p}}} \qquad ( 6 )$$

Taking natural logarithms of both sides of Equation (6) yields the following Equation (7):

$${log}_{e} \frac{\partial v ( t )}{\partial t} = {log}_{e} \frac{{I}_{c}}{{C}_{p}} - \frac{1}{{R}_{p} {C}_{p}} t \qquad ( 7 )$$

Here is considered a plane in which the horizontal axis represents time “t” and the vertical axis represents the following natural logarithm of the above-described time differential waveform of vt(t).

$${log}_{e} \frac{\partial {v}_{t} ( t )}{\partial t}$$

On this plane, Equation (7) is a straight line having the following gradient and intercept.

$$Gradient \text{:} - \frac{1}{{R}_{p} {C}_{p}} = - τ$$

$$Intercept \text{:} {log}_{e} \frac{\partial v ( t )}{\partial t}$$

Accordingly, with reference to FIG. 5, in the feature quantity calculating section 7, a natural logarithm of the differential coefficient of voltage waveform V(t) measured by the measuring section 6 is taken and plotted into this plane, the gradient of the line in the t-axis direction is estimated by the least squares method, and then the time constant τ is obtained as an absolute value of the reciprocal of the estimated gradient. The feature-quantity time constant τ contains information as to both resistance and capacitance components, thus making it possible to detect more detailed differences in electrical measurement of the skin.

Although the measurement of electrical characteristics of the skin has been mentioned in the description of the above first and second embodiments, the plurality of electrodes placed on the skin surface may also be used as electrodes for stimulating so-called acupuncture points. For example, the smaller the feature quantities at the measurement points 1 to “n” extracted in the feature quantity extracting section 7 are, the more easily the current flows through the measurement points, such sites of the skin being regarded as so-called acupuncture points. For more effective stimulation of such sites, it is also permissible that a current output from the current generator section 1 or the conduction current-applying electrode 3a to 3i is selected by the control section 20 based on the feature quantities. This enables beginners to effectively stimulate the acupuncture points.

The current dependency is observed in the electrical characteristics of the skin during the stimulation as described above or after the stimulation. However, by using nonlinear impedances for the electrical equivalent circuit of skin, it is possible to evaluate the electrical characteristics of the skin during the stimulation. Therefore, by extracting feature quantities that characterize the electrical characteristics of the skin as described in the foregoing first and second embodiments during the stimulation, it is possible to change the stimulant current generally in real time so that more efficient stimulation can be given to the skin.

### INDUSTRIAL APPLICABILITY

As described above, the skin conduction measuring apparatus according to the present invention is capable of eliminating the problems of the prior art as much as possible, making it possible to obtain more specific quantitative measurement results of higher reliability and reproducibility as compared with the prior art. Thus, the skin conduction measuring apparatus is useful for measuring electrical conductivities of the human body and using the measurement results to noninvasively and objectively evaluate differences of electrical characteristics of the skin in the medical field such as searching for the positions of acupuncture points or evaluating the health level and the.

1-9. (canceled)

10. A skin conduction measuring apparatus comprising:
a current generator section capable of generating pulsed electric currents;
an electrode system having a plurality of nonpolarizable electrodes to be placed on a plurality of different measurement points on a skin and functioning for conducting the currents output from the current generator section to the plurality of measurement points substantially simultaneously;
a plurality of current detectors for respectively detecting the currents conducted to the plurality of measurement points;
a measuring section for measuring the currents detected by the current detectors and for measuring voltages in the skin at the plurality of measurement points generated by the conduction of the electrode system;
a feature quantity extracting section for extracting a feature quantity that characterizes an electric current conductivity at each of the measurement points from a relationship between the current and the voltage measured by the measuring section;
a display section for displaying the feature quantity at each of the measurement points extracted by the feature quantity extracting section; and
a control section for generating control signals for the current generator section, the measuring section, and the feature quantity extracting section.

11. The skin conduction measuring apparatus according to claim 10, wherein the nonpolarizable electrodes are silver-silver chloride electrodes.

12. The skin conduction measuring apparatus according to claim 10, wherein the pulsed electric currents generated by the current generator section are bipolar pulse currents.

13. The skin conduction measuring apparatus according to claim 10, wherein the control section sets current values of the currents output from the current generator section to different values for the plurality of measurement points.

14. The skin conduction measuring apparatus according to claim 13, wherein the control section sets the currents output from the current generator section to such values that current dependency of the skin at the measurement points is not observed.

15. The skin conduction measuring apparatus according to claim 10, wherein, the feature quantity extracted by the feature quantity extracting section is associated with at least two of a resistance value Rp of a first resistor, a capacitance Cp of a capacitor, and a resistance value Rs of a second resistor under an assumption where an electrical equivalent circuit of the skin consists of the first resistor and the capacitor parallel-connected to each other and the second resistor series-connected to the parallel-connected first resistor and the capacitor.

16. The skin conduction measuring apparatus according to claim 15, wherein the feature quantity extracted by the feature quantity extracting section is an electrical conductivity G having a following relation with the resistance value Rp and the resistance value Rs:
G=1/(Rp+Rs).

17. The skin conduction measuring apparatus according to claim 15, wherein the feature quantity extracted by the feature quantity extracting section is a time constant τ having a following relation with the resistance value Rp and the capacitance Cp:
τ=1/(Rp·Cp).

18. The skin conduction measuring apparatus according to claim 13, wherein the control section sets the currents values of the currents output from the current generator section respectively for the plurality of measurement points based on the feature quantities respectively extracted by the feature quantity extracting section.
